(19) United States
(12) Patent Application Publication
(10) Pub. No.: US 2025/0186532 A1
(43) Pub. Date: Jun. 12, 2025

(54) SHINE MUSCAT JELLY HAVING HIGH SENSORY APPEAL AND EXCELLENT ANTIOXIDANT AND ANTITHROMBOTIC ACTIVITIES, AND METHOD OF PREPARING SAME

(71) Applicant: Andong National University Industry-Academic Cooperation Foundation, Andong-si (KR)

(72) Inventor: Ho Yong SOHN, Daegu (KR)

(73) Assignee: Andong National University Industry-Academic Cooperation Foundation, Andong-si (KR)

(21) Appl. No.: 18/955,701

(22) Filed: Nov. 21, 2024

(30) Foreign Application Priority Data
Dec. 6, 2023 (KR) 10-2023-0175347

(51) Int. Cl. A61K 36/87; A23L 21/15; A61K 35/744; A61K 36/888; A61K 38/39; A61P 7/02; A61P 39/06

(52) U.S. Cl. CPC A61K 36/87; A23L 21/15; A61K 35/744; A61K 36/888; A61K 38/39; A61P 7/02; A61P 39/06

(57) ABSTRACT

Proposed are a Shine Muscat jelly and a preparation method thereof using Shine Muscat, collagen, lactic acid bacteria, sugar, and konjac powder, specifically, sugar, konjac powder, low-molecular-weight collagen, and water are added to a trituration liquid of mature Shine Muscat, followed by boiling and cooling. Lactic acid bacteria powder is added thereto, and the resulting second mixture is transferred into a jelly mold and cooled again. With beneficial ingredients, this jelly possesses a high sensory appeal, and offers convenience, portability, and high consumer preference. The jelly exhibits excellent flavor, strong nitrite scavenging, and anti-thrombotic activities, making it a health functional food for improving blood circulation, preventing aging and cancer, and alleviating stress, which leads it to a very useful invention in the food industry. The jelly also will produce economic value by efficiently using Shine Muscat with limited storage capacity and creating high-value-added jelly from lower-quality fruits.

### CROSS REFERENCE TO RELATED APPLICATION

The present application claims priority to Korean Patent Application No. 10-2023-0175347, filed Dec. 6, 2023, the entire contents of which is incorporated herein for all purposes by this reference.

### STATEMENT REGARDING GOVERNMENT SPONSORED RESEARCH

This invention was supported by the Province of Gyeongsangbuk-do. [Project Management Institution: Industry-Academic Cooperation Foundation, Andong National University; Title of Research Business: 2023 Agriculture and Fisheries R&D Promotion Project; Title of Project: Development and Advancement of High-Value-Added Processed Products for the Revitalization of the Shine Muscat Industry in Gyeongsangbuk-do; Project Serial Number: 2023-0154; Contribution Rate: 100%]

### BACKGROUND OF THE DISCLOSURE

### 1. Field of the Disclosure

The present disclosure relates to a Shine Muscat jelly prepared with Shine Muscat, collagen, lactic acid bacteria, sugar, and konjac powder, and relates to a method of preparing the same. More specifically, the present disclosure relates to a Shine Muscat jelly prepared by adding sugar, konjac powder, low-molecular-weight collagen, and water to a trituration liquid of mature Shine Muscat, bringing a first mixture obtained to a boil and allowing the first mixture to cool, then adding lactic acid bacteria powder, mixing a second mixture obtained well, transferring the second mixture into a jelly mold, and allowing the second mixture to cool, and also the present disclosure relates to a method of preparing the same Shine Muscat jelly.

### 2. Description of the Related Art

Jelly refers to a dessert food made by boiling down juice or pulp and hardening the juice or pulp with an appropriate solidifying agent. A variety of solidifying agents such as agar, gelatin, carrageenan, and alginic acid are used. In particular, a sensory appeal can be enhanced by adding sugar and acid to the juice and pulp. Flavor can also be enhanced by using additives such as fresh ingredients and food coloring. The jelly can be made in various flavors and colors depending on the ingredients used. Currently, jelly is used as a decoration material for snacks, desserts, bread, and cakes.

Meanwhile, Shine Muscat is a grape variety developed in 1988 by the National Institute of Fruit and Tree Science (NIFT), which is a comprehensive research organization for agricultural and food industry technology under the Japanese Ministry of Agriculture, Forestry and Fisheries. Shine muscat is created by crossing “Akitsu-21” (Vitislabruscana) and “Hakunan” (Vitisvinifera). The scientific name of Shine Muscat is “Vitis vinifera”, the variety registration name is “Grape Agriculture No. 21 ( [symbol] )”, and the genome sequence registration code is disclosed at GCA_008326845.1. Shine Muscat has a thinner skin and no seeds than regular grapes, so Shine Muscat is easy to eat raw and has an excellent texture. Shine Muscat is moderately large in size, has a high sugar content (17 to 22 brix), and has a fresh and sweet aroma like mango. Thus, Shine Muscat is distributed as a high-quality fruit. However, recently, as domestic production has increased explosively, prices of Shine Muscat have been plummeting. As a result, there is a need to develop processed products of Shine Muscat such as in wine and juice.

Konjac refers to a jelly made from the starch of the root of Guyak herb, a type of taro. Konjac consists of 97% water, dietary fiber, and minerals. Glucomannan, which is contained in large quantities in konjac powder and a raw material of konjac, is a soluble dietary fiber which has the effect of suppressing a rise in blood sugar by delaying absorption of carbohydrates. In addition, konjac is low in calories and has an excellent obesity prevention effect. Konjac exhibits detox and anti-obesity effects because konjac absorbs and excretes ingested fat and cholesterol. In addition, konjac can prevent a rise in cholesterol levels by adsorbing bile acid in the digestive tract and can contribute to the prevention of cardiovascular disease.

Collagen (Kollagen or Collagen) is a polymer protein that makes up bones, skin, and cartilage, and accounts for almost 30% of proteins in the body. Since cells are mainly connected by collagen fibers, collagen can be said to be an essential protein in our body. To date, a total of 29 types of collagen proteins are known, including type I collagen. Collagen is a major component of skin and is being used in the development of inner beauty products to improve skin elasticity. Recently, low-molecular-weight collagen, which is easy to digest and absorb, is gaining popularity.

Lactic acid bacteria are representative probiotics and are known to be effective in improving intestinal health and immunity, improving digestive function, inhibiting the growth of harmful bacteria, and removing toxic substances. Thus, various types of lactic acid bacteria products are sold. Along with collagen, lactic acid bacteria are recognized as a representative source of inner beauty products.

To date, research related to Shine Muscat has mainly been related to its cultivation, storage, and distribution. There has been little research related to a physiological activity of Shine Muscat. Studies known until recently include “Efficiency Measurement and Factor Analysis for Management Stability of Shine Muscat Cultivation Farms” (Han Jinseong, 2023. Konkuk University (Doctoral Dissertation)), “Occurrence of Grape Bunch Rot Disease (Tentative Name) Caused by Aspergillus tubingensis on Shine Muscat” (Kim Youngsoo et al., 2019., Korean Journal of Plant Pathology, Vol. 25, pp. 220-225), “Effects of Post-harvest Treatments for Improving Storage and Distribution of ‘Campbell Early’ and ‘Shine Muscat’ Grapes for Export” (Kim Seongju, 2020, Chungnam National University (Doctoral Dissertation)), “Establishment of Conditions for Mass Propagation and Virus Elimination to Produce Virus-free Plants of Shine Muscat (Vitis vinifera “Shine Muscat”)” (Kim Sihong, 2023, Kangwon National University (Doctoral Dissertation)), “Efficiency Measurement and Factor Analysis of Shine Muscat Grape Cultivation Farms: Focusing on Farms in Gyeongbuk Province” (Im Seungjoo et al., 2019, Journal of Food Distribution Research, pp. 49-72), “Effects of Cluster Thinning and Growth Regulators on ‘Shine Muscat’ Grapes” (Shin Hyunwook, 2019, Kyungpook National University (Master's Thesis)), and “Characterization of Mannitol and Exopolysaccharide-Producing Lactic Acid Bacteria from Kimchi” (Kang Yoonji, 2023, Gyeongsang National University (Master's Thesis)). However, there are no known studies on Shine Muscat jelly, which has an excellent sensory appeal and antithrombotic activity.

Disclosed patent literature related to Shine Muscat includes, for example, Korean Patent No. 10-2287416, titled “Fruit Extract Complex of Vitis vinifera, Pyrus malus, Prunus mume and Carica papaya and Cosmetic Composition Containing of Same”, Korean Patent Application Publication No. 10-2023-0070717, titled “Manufacturing Method of Beverage Containing Vitis vinifera and Blueberry”, Korean Patent Application Publication No. 10-2023-0078852, titled “Shine Muscat Powder, Shine Muscat Filling, Shine Muscat Bread and Method for Manufacturing Same”, Korean Patent Application Publication No. 10-2023-0111395, titled “Method for Manufacturing Fruit Powder and Fruit Filling and Bread Comprising Fruit Powder”, and Korean Patent No. 30-1138226, titled “Color Chart for Determining Ripeness of Grape Shine Muscat”. However, to date, there has been no known research on Shine Muscat jelly, which has a high sensory appeal and excellent antioxidant, and antithrombotic activities.

### RELATED ART DOCUMENT

### Patent Document

(Patent document 0001) KR 10-2023-0078852 A

### SUMMARY OF THE DISCLOSURE

The present disclosure is devised to solve the problems of the conventional art as described above. The present disclosure is to provide a Shine Muscat jelly prepared with Shine Muscat, collagen, lactic acid bacteria, sugar, and konjac powder, and to provide a method of preparing the same Shine Muscat jelly.

To address the problems described above, the present disclosure provides a method of preparing a Shine Muscat jelly, the method including: (1) obtaining a trituration liquid of mature Shine Muscat; (2) adding 5% to 15% by weight of sugar, 1% to 3% by weight of konjac powder, 0.5% to 1.5% by weight of low-molecular-weight collagen, and 50% to 60% by weight of water to 20% to 40% by weight of the trituration liquid, bringing a first mixture obtained to a boil for 2 to 5 minutes, and allowing the first mixture to cool; and (3) adding and 0.5% to 1.5% by weight of lactic acid bacteria powder to the first mixture and mixing a second mixture obtained, then transferring the second mixture into a jelly mold and allowing the second mixture to cool.

The Shine Muscat may preferably have a sugar content of 15 to 18 Brix.

In addition, the present disclosure provides a Shine Muscat jelly prepared by the method of the present disclosure.

The Shine Muscat jelly may preferably exhibit antioxidant activity and antithrombotic activity.

The Shine Muscat jelly of the present disclosure contains the beneficial ingredients of Shine Muscat, and possesses a high sensory appeal, high portability, accessibility, and high preference. The Shine Muscat jelly has an excellent flavor. In addition, the Shine Muscat jelly exhibits strong nitrite scavenging activity and anti-thrombotic activity, thereby the Shine Muscat jelly is also expected to be used as a health functional food for improving blood circulation, inhibiting aging, inhibiting carcinogenesis, and relieving stress. Thus, Shine Muscat jelly is a very useful invention in the food industry. In addition, economic feasibility is expected to increase in terms of efficient use of Shine Muscat with a low storage capacity and production of high value-added jelly using low-quality fruits.

### BRIEF DESCRIPTION OF THE DRAWINGS

FIG. 1 shows photographs of Shine Muscat jellies prepared with varying concentrations of ingredients, 1: Shine Muscat 15% by weight, sugar 5% by weight, konjac powder 1.7% by weight, and water 78.3% by weight, 2: Shine Muscat 25% by weight, sugar 5% by weight, konjac powder 1.7% by weight, and water 68.3% by weight, 3: Shine Muscat 35% by weight, sugar 0% by weight, konjac powder 1.7% by weight, and water 63.3% by weight, 4: Shine Muscat 25% by weight, sugar 10% by weight, konjac powder 1.7% by weight, and water 63.3% by weight, 5: Shine Muscat 30% by weight, sugar 10% by weight, konjac powder 1.7% by weight, and water 58.3% by weight;

FIG. 2 shows results of measuring the physical properties of Shine Muscat jellies prepared with varying concentrations of ingredients, 1: Shine Muscat 15% by weight, sugar 5% by weight, konjac powder 1.7% by weight, and water 78.3% by weight, 2: Shine Muscat 25% by weight, sugar 5% by weight, konjac powder 1.7% by weight, and water 68.3% by weight, 3: Shine Muscat 35% by weight, sugar 0% by weight, konjac powder 1.7% by weight, and water 63.3% by weight, 4: Shine Muscat 25% by weight, sugar 10% by weight, konjac powder 1.7% by weight, and water 63.3% by weight, 5: Shine Muscat 30% by weight, sugar 10% by weight, konjac powder 1.7% by weight, and water 58.3% by weight;

FIG. 3 shows photographs showing (1) a Shine Muscat jelly prepared by adding 1% by weight of collagen powder to a mixture of 30% by weight of Shine Muscat, 10% by weight of sugar, 1.7% by weight of konjac powder, and 57.3% by weight of water, (2) a Shine Muscat jelly prepared by adding 1% by weight of lactic acid bacteria powder to a mixture of 30% by weight of Shine Muscat, 10% by weight of sugar, 1.7% by weight of konjac powder, and 57.3% by weight of water, and (3) a Shine Muscat jelly prepared by adding 1% by weight of collagen powder and 1% by weight of lactic acid bacteria powder to a mixture of 30% by weight of Shine Muscat, 10% by weight of sugar, 1.7% by weight of konjac powder, and 56.3% by weight of water; and

FIG. 4 shows photographs showing jellies prepared using (1) a Shine Muscat trituration liquid, (2) spray-dried Shine Muscat powder, and (3) spray-dried grape (Campbell Early) powder.

### DESCRIPTION OF THE PREFERRED EMBODIMENTS

Hereinafter, the present disclosure will be described in detail.

To efficiently use Shine Muscat, the inventors of the present disclosure washed and cleaned Shine Muscats, then triturated the Shine Muscats and extracted their juice. Then, the inventors added collagen, sugar, and konjac powder thereto, brought a first mixture obtained to a boil, and allowed the first mixture to cool. Next, the inventors added the mixed lactic acid bacteria powder to the first mixture and mixed a second mixture obtained well, transferred the second mixture into a jelly mold, and allowed the second mixture to cool, thereby Shine Muscat jelly foods (hereinafter, Shine Muscat jelly foods will be described as Shine Muscat jellies) were prepared. The Shine Muscat jellies prepared of the present disclosure had a high sensory appeal. In addition, The Shine Muscat jellies prepared exhibited strong nitrite scavenging ability and anti-thrombotic activity. Due to the addition of collagen and lactic acid bacteria, these Shine Muscat jellies may possess health functionality.

Thus, the present disclosure provides a method of preparing a Shine Muscat jelly, the method including: (1) obtaining a trituration liquid of mature Shine Muscat, (2) adding 5% to 15% by weight of sugar, 1% to 3% by weight of konjac powder, 0.5% to 1.5% by weight of low-molecular-weight collagen, and 50% to 60% by weight of water to 20% to 40% by weight of the trituration liquid, bringing a first mixture obtained to a boil for 2 to 5 minutes, and allowing the first mixture to cool, and (3) adding and 0.5% to 1.5% by weight of lactic acid bacteria powder to the first mixture and mixing a second mixture obtained, then transferring the second mixture into a jelly mold and allowing the second mixture to cool.

In one preferred embodiment, the method of preparing a Shine Muscat jelly of the present disclosure may include: (1) washing and cleaning mature Shine Muscat and obtaining a trituration liquid of the mature Shine Muscat, (2) adding 10% by weight of sugar, 1.7% by weight of konjac powder, 1% by weight of low-molecular-weight collagen, and 56.3% by weight of water to 30% by weight of the trituration liquid of the Shine Muscat, and then bringing a first mixture obtained to a boil at a temperature of 100° C. for 2 to 5 minutes and then allowing the first mixture to cool to a temperature of 60° C., and (3) adding 1% by weight of mixed lactic acid bacteria powder to the first mixture and mixing a second mixture obtained well, then transferring the second mixture into a jelly mold and allowing the second mixture to cool.

The Shine Muscat preferably has a sugar content of 15 to 18 Brix.

The Shine Muscat is mature Shine Muscat and preferably has a unique mango aroma. Since the trituration liquid of a Shine Muscat is used, the Shine Muscat for use does not need to have a soft and thin-skinned texture that consumers value.

Shine Muscat jelly according to the method of preparing a Shine Muscat jelly of the present disclosure has excellent portability, accessibility, and storage. The Shine Muscat jelly of the present disclosure has a high sensory appeal and exhibits nitrite scavenging ability and antithrombotic activity.

Thus, the present disclosure provides a Shine Muscat jelly prepared by the method of the present disclosure.

The Shine Muscat jelly preferably exhibits antioxidant activity and antithrombotic activity.

With the antioxidant activity, the Shine Muscat jelly may effectively remove reactive oxygen species exposed to human cells. For example, the antioxidant activity may be associated with inhibition of skin aging, anti-cancer activity, and prevention of cardiovascular disease.

Thus, the present disclosure provides antioxidant health functional foods by using the Shine Muscat jelly of the present disclosure with the nitrite scavenging ability.

In addition, the antithrombotic activity may be associated with various diseases related to thrombosis. These diseases, for example, may include arterial thrombosis, which could cause acute myocardial infarction, chest pain, shortness of breath, loss of consciousness, ischemic stroke, hemorrhagic stroke, headache, motor abnormalities, sensory abnormalities, personality changes, blurred vision, epileptic seizures, pulmonary thrombosis, deep vein thrombosis, lower extremity edema, pain, and acute peripheral arterial occlusion, and may include venous thrombosis, which could cause deep vein thrombosis, portal vein thrombosis, acute renal vein occlusion, cerebral venous sinus thrombosis, and central retinal vein occlusion.

Thus, the present disclosure provides anti-thrombotic health functional foods to improve blood circulation by using the Shine Muscat jelly of the present disclosure.

The health functional foods containing the Shine Muscat jelly of the present disclosure may include, for example, various foods, beverages, tea, vitamin complexes, and health supplements. The health functional foods may be in the form of powders, granules, tablets, capsules, or beverages.

In addition to containing the Shine Muscat jelly as an essential ingredient in an indicated ratio, the health functional foods of the present disclosure may contain food supplements acceptable in food science, such as natural carbohydrates and various flavoring agents, as additional ingredients.

The natural carbohydrates may include common sugars such as monosaccharides (for example, glucose and fructose), disaccharides (for example, maltose and sucrose), and polysaccharides (for example, dextrin and cyclodextrin), and sugar alcohols such as xylitol, sorbitol, and erythritol.

The flavoring agents may include natural flavoring agents such as thaumatin, rebaudioside A, or stevia such as glycyrrhizin, and synthetic flavoring agents such as saccharin and aspartame. The amount of natural carbohydrates is generally about 1 to 20 g, preferably about 5 to 12 g, per 100 ml of the health functional foods of the present disclosure. In addition to the food supplements, the health functional foods of the present disclosure may include various nutritional supplements, vitamins, minerals, flavoring agents such as synthetic and natural flavors, colorants and thickening agents, pectic acid and its salts, alginic acids and its salts, organic acids, protective colloidal thickeners, pH adjusters, stabilizers, preservatives, glycerin, alcohol, and carbonating agents used in carbonated drinks.

Hereinafter, the present disclosure will be described in more detail through specific examples. The following examples describe preferred examples of the present disclosure, and the scope of the present disclosure is not limited by the matters described in the following examples.

### EXAMPLES

### Example 1: Preparation of Shine Muscat Jellies

Shine Muscats used in Example 1 were Shine Muscats grown in Sangju, Gyeongbuk, South Korea in 2022 and stored in refrigeration. Washed and selected Shine Muscats were triturated and then extracted with a screw press to obtain a trituration liquid thereof. Then, various concentrations of sugar, konjac powder, and water were added to the obtained trituration liquid, and a first mixture obtained was mixed. Next, the first mixture was brought to a boil at a temperature of 100° C. for 2 to 5 minutes. Thereafter the first mixture was transferred into a jelly mold and allowed to cool at room temperature.

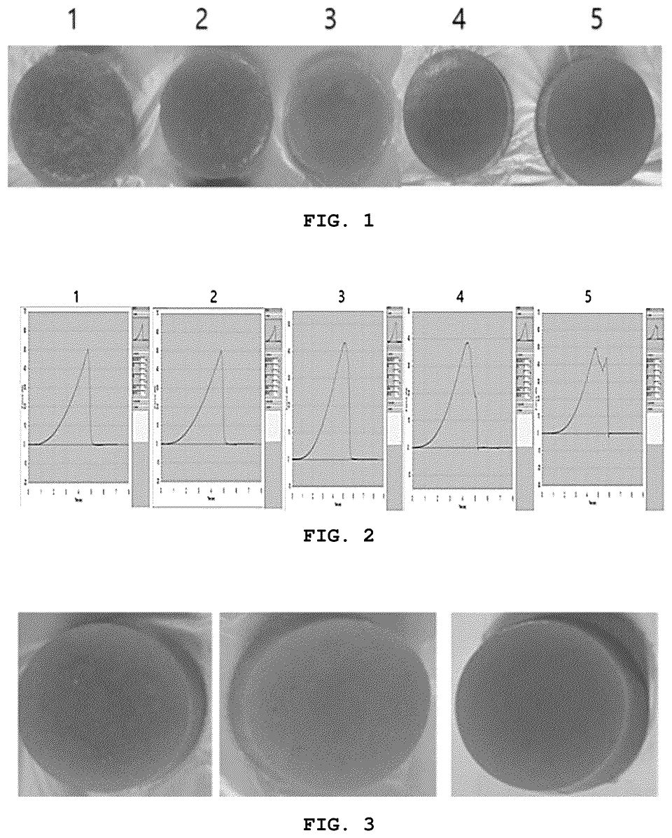

First, to prepare Shine Muscat jellies with a high sensory appeal, various concentrations of trituration liquids of the Shine Muscats, sugar, and konjac powder were added. The composition table and photos of the five types of jellies prepared are shown in Table 1 and FIG. 1.

TABLE 1
Preparation of Shine Muscat Jellies with
Varying Concentrations of Ingredients

| | Shine Muscat | | Konjac | |
|---|---|---|---|---|
| Division | trituration liquid | Sugar | powder | Water |
| Composition 1 | 15 | 5 | 1.7 | 78.3 |
| Composition 2 | 25 | 5 | 1.7 | 68.3 |
| Composition 3 | 35 | 0 | 1.7 | 63.3 |
| Composition 4 | 25 | 10 | 1.7 | 63.3 |
| Composition 5 | 30 | 10 | 1.7 | 58.3 |

As a result of measuring the sensory appeal and chewiness of the jellies prepared, as shown in Table 2 and FIG. 1, a jelly prepared by adding 30% by weight of trituration liquid of a Shine Muscat, 10% by weight of sugar, 1.7% by weight of konjac powder, and 58.3% by weight of water was the best in taste, aroma, and chewiness. In particular, in the case of the composition, as can be seen from force change measurement results using a jelly physical property measuring device in FIG. 2, the jelly had the best texture as the jelly exhibited a soft chewiness and a sticking property to the teeth moderately. At this time, the sensory appeal evaluation was conducted by six professional researchers in the Department of Food and Nutrition, evaluating each taste, aroma, texture, and overall preference using a 7-point scale (7 points: Best, up to 3.5 points: So-so, up to 1 point: worst). The chewiness of the jelly was measured using Texture analyzer FRTS-50N-I (Imada Co. LTD., Japan) and Force Recorder Professional Software (FRTS ver. 1.0), and after three repeated measurements, an average value was obtained. Hardness was measured using a disk probe, compression speed was set at 2.0 mm/see, and compression displacement was set at 10 mm.

TABLE 2
Sensory Appeal and Chewiness Evaluation of Shine Muscat
Jellies with Varying Concentrations of Ingredients

| | | | | Overall | Hardness |
|---|---|---|---|---|---|
| Division | Flavor | Aroma | Texture | preference | (rheometer) |
| Composi- | 3.6 ± 1.2 | 4.2 ± 0.4 | 5.0 ± 0.3 | 4.7 ± 0.5 | 50.14 |
| tion 1 | | | | | |
| Composi- | 4.7 ± 0.2 | 4.9 ± 0.1 | 5.0 ± 0.2 | 4.9 ± 0.8 | 50.17 |
| tion 2 | | | | | |
| Composi- | 4.0 ± 0.3 | 5.2 ± 0.2 | 5.0 ± 0.5 | 4.7 ± 0.8 | 43.09 |
| tion 3 | | | | | |
| Composi- | 5.0 ± 0.1 | 5.0 ± 0.7 | 5.0 ± 0.3 | 5.0 ± 0.8 | 38.47 |
| tion 4 | | | | | |
| Composi- | 5.7 ± 0.3 | 5.4 ± 0.4 | 5.0 ± 0.2 | 5.7 ± 0.8 | 49.20 |
| tion 5 | | | | | |

### Example 2: Preparation of Shine Muscat Jellies with Lactic Acid Bacteria and Collagen Added

To provide additional health functionality, various concentrations of lactic acid bacteria and collagen were added to the mixture, which was prepared with a composition of 30% by weight of trituration liquid of Shine Muscat, 10% by weight of sugar, 1.7% by weight of konjac powder, and 58.3% by weight of water, as determined in Example 1. Thereby, Shine Muscat jellies were prepared. As a result, as shown in FIG. 3, it was confirmed that the addition of 1% by weight of lactic acid bacteria and 1% by weight of collagen was most preferable. In this case, there was no decrease in sensory appeal such as taste, aroma, or chewiness. However, when lactic acid bacteria and collagen were added simultaneously, an increase in yellowness appeared.

### Example 3: Preparation of Shine Muscat Jellies with Lactic Acid Bacteria and Collagen Added

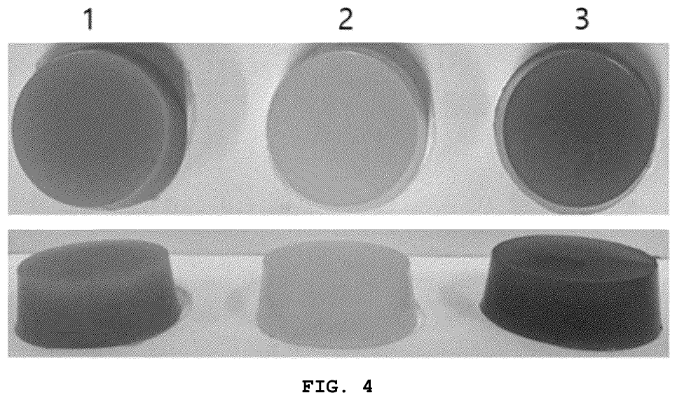

The properties of the Shine Muscat jelly (hereinafter this jelly may also be described as Shine Muscat trituration liquid jelly) (composition: 30% by weight of trituration liquid of a Shine Muscat, 10% by weight of sugar, 1.7% by weight of konjac powder, 1% by weight of low-molecular-weight collagen, 1% by weight of mixed lactic acid bacteria powder, 58.3% by weight of water) finally determined in Example 2, a commercially available jelly made from Shine Muscat powder (hereinafter this jelly may also be described as Shine Muscat powder jelly), and a commercially available jelly made from grape (Campbell's Early) powder (hereinafter, this jelly may also be described as grape powder jelly) were compared and evaluated. At this time, considering the moisture content of the trituration liquid of the Shine Muscat and the concentration of excipients during spray drying, 10% by weight of Shine Muscat powder and 10% by weight of grape (Campbell's Early) powder were added in each corresponding case (Table 3 and FIG. 4).

TABLE 3
Jelly Preparation Using Shine Muscat Trituration Liquid and Powder

| | Shine Muscat | Shine | | | | | | |
|---|---|---|---|---|---|---|---|---|
| | trituration | Muscat | Grape | | Konjac | Low-molecular- | lactic acid | |
| Division | liquid | powder | powder | Sugar | powder | weight collagen | bacteria | Water |
| Shine Muscat | 30 | — | — | 10 | 1.7 | 1.0 | 1.0 | 56.3 |
| trituration | | | | | | | | |
| liquid jelly | | | | | | | | |
| Shine Muscat | — | 10 | — | 10 | 1.7 | 1.0 | 1.0 | 76.3 |
| powder jelly | | | | | | | | |
| Grape powder | — | — | 10 | 10 | 1.7 | 1.0 | 1.0 | 76.3 |
| jelly | | | | | | | | |

As shown in FIG. 3, when the trituration liquid of the Shine Muscat was added, the Shine Muscat trituration liquid jelly showed a darker yellow color than the commercially available Shine Muscat powder jelly, and in the case of the commercially available grape powder jelly, a pink jelly was prepared. Color difference analysis results for the three types of jellies are shown in Table 4. When the trituration liquid of the Shine Muscat was added, brightness and yellowness of the Shine Muscat trituration liquid jelly were significantly higher than in other cases. In addition, in terms of functionality, the case where the trituration liquid of the Shine Muscat was added showed the highest functionality. Although this result may incur some costs, it suggested that it was preferable to use the trituration liquid of the Shine Muscat to prepare a Shine Muscat jelly with excellent functionality.

At this time, color difference analysis was measured using a Hunter Color Difference meter (Super color SP80 Colorimeter, Tokyo Denshoku Co., Japan). Brightness (white 100 to 0 black), redness (red 100 to −80 green), and yellowness (yellow 70 to −80 black) were measured. The standard chromaticity of the standard whiteboard was set with an L value of 92.44, a value of −0.06 for a, and a value of 1.35 for b. Each sample was measured three times, and an average value was calculated and expressed. Color difference (ΔE) was calculated using the following equation.

$$( Δ E = \sqrt{{( Δ L )}^{2} + {( Δ a )}^{2} + {( Δ b )}^{2}} )$$

TABLE 4
Color Difference Analysis of Shine
Muscat Jelly of Present Disclosure

| | Color difference analysis | | | |
|---|---|---|---|---|
| | Brightness | Redness | Yellowness | Color |
| Division | (L) | (a) | (b) | difference |
| Shine Muscat | 29.89 ± 0.01 | −1.52 ± 0.00 | 6.24 ± 0.07 | 62.70 ± 0.01 |
| trituration | | | | |
| liquid jelly | | | | |
| Shine Muscat | 24.43 ± 0.08 | −1.59 ± 0.04 | 1.63 ± 0.04 | 67.98 ± 0.08 |
| powder jelly | | | | |
| Grape powder | 18.00 ± 0.00 | 3.45 ± 0.07 | 1.99 ± 0.14 | 74.47 ± 0.00 |
| jelly | | | | |

### Example 4: Antithrombotic Activity of Shine Muscat Jelly of Present Disclosure

An antithrombotic activity was measured for the three types of jellies prepared in Example 3, and the results are shown in Table 5. At this time, the blood coagulation inhibitory activity evaluation method was in accordance with the previously reported method (Sohn et al., 2004. Kor. J. Pharmacogn 35. 52-61; Kwon et al., 2004. J. Life Science, 14. 509-513; Ryu et al. 2010. J. Life Science, 20. 922-928). Experiments were conducted to measure thrombin time, prothrombin time, and activated Partial Thromboplastin Time (aPTT). For plasma, commercially available control plasma (MD Pacific Technology Co., Ltd, Huayuan Industrial Area, China) was used. The thrombin time, prothrombin time, and aPTT measurements were performed as follows.

### Thrombin Time

At a temperature of 37° C., 50 μl of 0.5 U thrombin (Sigma Co., USA), 50 μl of 20 mM CaCl₂), and 10 μl of sample extracts of various concentrations were mixed in a tube of Amelung coagulometer KC-1A (Japan), and reacted for 2 minutes. Thereafter, 100 μl of plasma was added thereto. Afterward, the time until the plasma coagulated was measured. Aspirin (Sigma Co., USA) was used as a control. As a solvent control, DMSO was used instead of the sample extracts. In the case of DMSO, the coagulation time was 32.1 seconds. The thrombin inhibition effect was expressed as an average value of experiments repeated more than three times. A thrombin inhibitory activity was expressed as the coagulation time upon sample addition divided by the coagulation time upon the solvent control addition.

### Prothrombin Time

At a temperature of 37° C., 70 μl of standard plasma (MD Pacific Co., China) and 10 μl of sample solutions of various concentrations were added into the tube of Amelung coagulometer KC-1A (Japan) and heated for 3 minutes. Thereafter, 130 μl of PT reagent was added thereto. Afterward, the time until the plasma coagulated was expressed as an average value of the experiments repeated three times. Aspirin (Sigma Co., USA) was used as a control. As a solvent control, DMSO was used instead of the sample extracts. In the case of DMSO, the coagulation time was 18.1 seconds. A prothrombin inhibitory activity was expressed as the coagulation time upon sample addition divided by the coagulation time upon the solvent control addition.

Activated Partial Thromboplastin Time (aPTT)

At a temperature of 37° C., 100 μl of plasma and 10 μl of sample extracts of various concentrations were added to the tube of Amelung coagulometer KC-1A (Japan) and heated for 3 minutes. Thereafter, 50 μl of aPTT reagent (Sigma, ALEXIN™) was added thereto. Then, the final mixture was cultured at a temperature of 37° C. again for 3 minutes. Next, 50 μl CaCl₂) (35 mM) was added thereto. Afterward, the time until the plasma coagulated was measured. As a solvent control, DMSO was used instead of the sample extracts, and in this case, the coagulation time was 55.1 seconds. The results of aPTT were expressed as an average value of three repeated experiments. A blood coagulation factor inhibitory activity was expressed as the aPTT upon sample addition divided by the aPTT upon the solvent control addition.

TABLE 5
Blood Coagulation Inhibitory Activity of
Shine Muscat Jelly of Present Disclosure

| Control/ | Concentra- | *Blood coagulation inhibitory activity | | |
|---|---|---|---|---|
| Extract/ | tion | Thrombin | Prothrombin | |
| Fraction | (mg/ml) | time | time | aPTT |
| Solvent | — | 1.00 ± 0.00 | 1.00 ± 0.00 | 1.00 ± 0.001 |
| control | | | | |
| (DMSO) | | | | |
| Aspirin | 1.5 | 1.26 ± 0.00 | 1.30 ± 0.02 | 1.33 ± 0.02 |
| Shine Muscat | | 1.84 ± 0.02 | 1.24 ± 0.03 | 1.94 ± 0.01 |
| trituration | | | | |
| liquid jelly | | | | |
| Shine Muscat | | 2.20 ± 0.04 | 1.04 ± 0.03 | 1.32 ± 0.05 |
| powder jelly | | | | |
| Grape powder | | 1.31 ± 0.00 | 1.00 ± 0.01 | 1.63 ± 0.03 |
| jelly | | | | |

*Blood coagulation inhibitory activity: blood coagulation time upon sample addition/blood coagulation time upon DMSO addition

As shown in Table 5, it was confirmed in the measurements that aspirin (antithrombotic drug brand name: Protect) served to extend thrombin time, prothrombin time, and aPTT by 1.26 times, 1.30 times, and 1.33 times, respectively, at a concentration of 1.5 mg/ml, exhibiting excellent antithrombotic activity. The Shine Muscat trituration liquid jelly served to extend thrombin time, prothrombin time, and aPTT by 1.84 times, 1.24 times, and 1.94 times, respectively, exhibiting excellent antithrombotic activity. Meanwhile, the Shine Muscat powder jelly prepared by using Shine Muscat hot air-dried powder served to extend thrombin time by approximately 2.2 times compared to the untreated group, exhibiting excellent thrombin inhibitory activity, but the extension of prothrombin time and aPTT was relatively short. In addition, the grape powder jelly prepared by using grape powder (Campbell Early's hot air-dried powder) served to extend thrombin time and prothrombin time by 1.31 times and 1.00 times, respectively compared to the untreated group, which meant a relatively short extension, but aPTT was extended by 1.63 times compared to the untreated group. Thus, the inhibitory effect of grapes on endogenous blood clot formation was confirmed. However, total inhibition of blood coagulation factors, prothrombin, and thrombin is required for antithrombotic activity given the cascade reaction of thrombus formation. Accordingly, it was confirmed that the most ideal antithrombotic activity was exhibited in the jelly prepared by adding a trituration liquid of a Shine Muscat.

### Example 5: Nitrite Scavenging Activity of Shine Muscat Jelly of Present Disclosure

A nitrite scavenging activity was measured for the three types of jellies prepared in Example 3, and the results are shown in Table 6. The nitrite scavenging activity may prevent cancer caused by nitrite and also contribute to antioxidant activity.

To measure the nitrite scavenging ability, a sample solution was added to the nitrite solution (1 mM), and 0.1 N HCl was further added thereto to adjust the pH of the mixture to 1.2. Thereafter, the mixture was reacted at a temperature of 37° C. for 1 hour, and then Griess reagent (Sigma Co., USA) was added, and the resulting solution was mixed. After leaving the resulting solution at room temperature for 15 minutes, the amount of remaining nitrite was measured by measuring absorbance at a wavelength of 520 nm. The nitrite scavenging ability (%) was calculated using the following equation.

TABLE 6
Evaluation of Nitrite Scavenging Activity
of Shine Muscat Jelly of Present Disclosure

| | Nitrite scavenging ability |
|---|---|
| Division | (0.2 mg/ml) |
| Shine Muscat trituration liquid jelly | 23.9 ± 2.8 |
| Shine Muscat powder jelly | 3.6 ± 2.1 |
| Grape powder jelly | 3.8 ± 3.6 |
| Vitamin C ((0.2 mg/ml)) | 62.5 ± 0.1 |

As shown in Table 6, the jelly prepared by adding a trituration liquid of a Shine Muscat showed a very strong nitrite scavenging ability, unlike other jellies. The Shine Muscat trituration liquid jelly exhibited weaker activity than vitamin C, which was used as a control, but exhibited more powerful activity than other jellies by 6 times or more. In addition, it has been reported that nitrite is also involved in the formation of blood clots (Martinez, M., et al., 2013. Free Radic. Biol. Med. 65, 411-418, Matarrese, P., et al., 2005. FASEB J. 19, 416-418, Wang, L., et al., 2017. Sci. Rep. 7, 12429). From this, it is believed that the jelly prepared by adding the trituration liquid of a Shine Muscat of the present disclosure will contribute to anti-thrombotic and antioxidant activities due to nitrite removal and inhibition of thrombogenic factors and enzymes. Meanwhile, the DPPH anion scavenging ability, ABTS cation scavenging ability, and reducing power evaluation results for the three types of jellies exhibited no significant differences and overall exhibited weak active ion scavenging ability.

What is claimed is:

1. A method of preparing Shine Muscat jelly, the method comprising:
(1) obtaining a trituration liquid of mature Shine Muscat;
(2) adding 5% to 15% by weight of sugar, 1% to 3% by weight of konjac powder, 0.5% to 1.5% by weight of low-molecular-weight collagen, and 50% to 60% by weight of water to 20% to 40% by weight of the trituration liquid, bringing a first mixture obtained to a boil for 2 to 5 minutes, and allowing the first mixture to cool; and
(3) adding 0.5% to 1.5% by weight of lactic acid bacteria powder to the first mixture and mixing a second mixture obtained, then transferring the second mixture into a jelly mold and allowing the second mixture to cool.

2. The method of claim 1, wherein the Shine Muscat has a sugar content of 15 to 18 Brix.

3. A Shine Muscat jelly prepared by any method of claim 1.

4. The Shine muscat jelly of claim 3, wherein the Shine Muscat jelly exhibits antioxidant activity and antithrombotic activity.

5. A Shine Muscat jelly prepared by any method of claim 2.

6. The Shine muscat jelly of claim 5, wherein the Shine Muscat jelly exhibits antioxidant activity and antithrombotic activity.
